(19) United States
(12) Patent Application Publication
(10) Pub. No.: US 2007/0213794 A1
(43) Pub. Date: Sep. 13, 2007

(54) "Cool Down" vinyl, freezable gel-filled neck collar

(76) Inventor: Diana Lyn (Gieb) Lauver, Sedley, VA (US)

Correspondence Address: Diana Lauver, 16205 Ivor Road, Sedley, VA, 23878, US

(21) Appl. No.: 11/373,594

(22) Filed: Mar. 13, 2006

(51) Int. Cl. A61F 7/00

(52) U.S. Cl. 607/109

(57) ABSTRACT

“Cool Down” is a safe, easy-to-use neck collar used to help reduce and regulate body temperature. “Cool Down” works quickly and comfortably. The soft vinyl exterior is lightweight and hypoallergenic. The non-toxic gel interior is flexible and freezes quickly. The interior and exterior work together to provide quick and comfortable relief from hot flashes and help prevent of hyperthermia, during exercise.

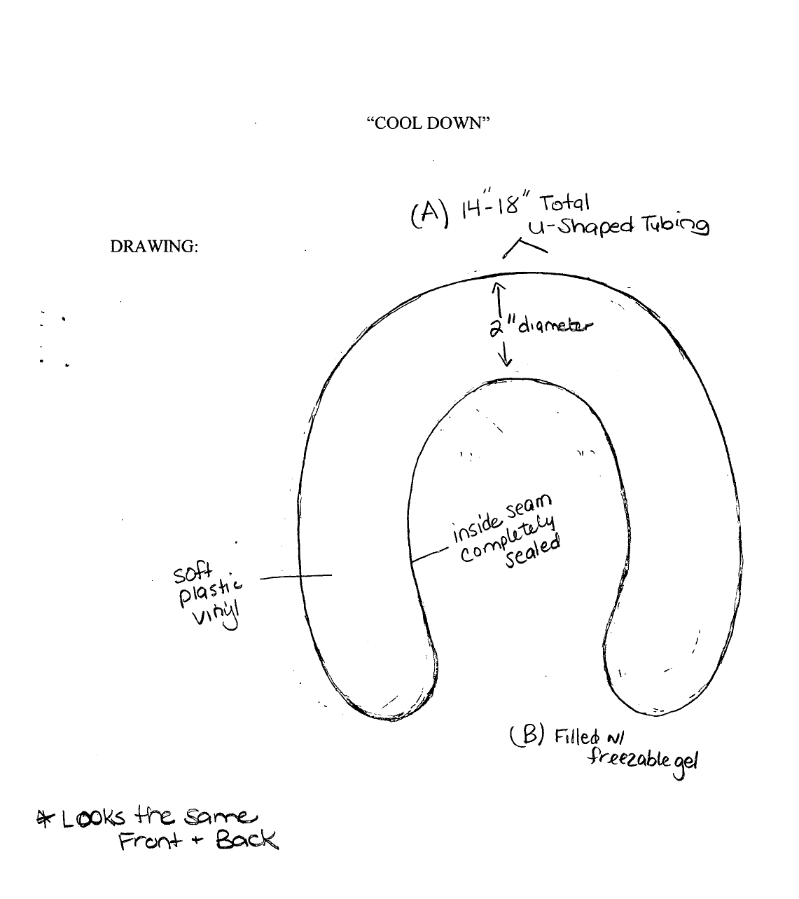

### BACKGROUND

The “Cool Down” Freezable Neck Collar is a safe and effective way to quickly relieve hot flashes and help prevent hyperthermia.

A hot flash is defined as a sudden, brief sensation of heat. 75-80% of Menopausal women suffer from hot flashes, making hot flashes the most prevalent symptom of menopause. Hot flashes, while not life threatening, are very uncomfortable. The most commonly used treatment for women with menopausal symptoms has been hormone replacement treatment. Recently researchers have found no benefits to support the continued use of HRT and a direct correlation in HRT and the onset of heart conditions. The risks seem to outweigh the benefits of HRT. It is essential to have practical and safe alternatives to relieve hot flashes.

Hyperthermia is abnormally high body temperature. Hyperthermia (or over-heating) can be brought on by vigorous exercise or exercise done in hot temperatures. It can lead to mild conditions, such as heat exhaustion, as well as life-threatening heat stroke. To prevent over-heating during exercise, body temperature should be regulated.

“Cool Down” is a quick and comfortable way to reduce and regulate body temperature.

### DESCRIPTION OF PREFERRED EMBODIMENT/SPECIFICATIONS

Exterior (A on Diagram):

U-shaped tubing, made of soft, lightweight plastic vinyl.
Total length: Small 14 inches long
   Large 18 inches long
Total width: Small 2 inch diameter
   Large 2 inch diameter
Can be produced in a variety of colors.
   Interior (B on Diagram)—Inside the Tubing

Non-Toxic Freezable, Flexible Gel

Chemical Compound:
Water (69.8%), 1.2 Propanediol (29.8%), Triethanolamine mixure (0.2%), Preservative (0.2%), Acrylic Polymer (0.2%)

### DESCRIPTION

“Cool Down” is a product that can be easily and inexpensively created. Plastic (in a variety of colors) is used to form 18″ (for large) and 14″ (for small) long by 2″ diameter, soft, vinyl, U-shaped tubes. One end of the tube is permanently sealed, with plastic. The tube is 99% filled with a non-toxic freezable gel. The other end of the tube is then permanently sealed. The additional 1% allows the neck collar to be flexible.

1. “Cool Down” is a non-toxic, freezable neck collar that is used to quickly reduce and regulate body temperature. The soft, lightweight vinyl exterior and U-shaped tubular design help it fit comfortably and discreetly around the neck. The non-toxic gel inside is flexible and freezable, providing even more comfort and discretion. “Cool Down” is a safe, practical alternative to medications for the relief of hot flashes and an effective way to regulate body temperature during exercise.
